(19) United States
(12) Patent Application Publication
(10) Pub. No.: US 2015/0142332 A1
(43) Pub. Date: May 21, 2015

(54) HEALTH CARE SYSTEM AND METHOD USING STRESS INDEX ACQUIRED FROM HEART RATE VARIATION

(71) Applicant: KOREA ELECTRONICS TECHNOLOGY INSTITUTE, Seongnam-si (KR)

(72) Inventors: Ki Man JEON, Seongnam-si (KR); Hyun Woo KIM, Seoul (KR); Ha Joong CHUNG, Seongnam-si (KR)

(21) Appl. No.: 14/605,948

(22) Filed: Jan. 26, 2015

Related U.S. Application Data
(62) Division of application No. 13/536,404, filed on Jun. 28, 2012.

(51) Int. Cl. G06F 19/00; A61B 5/0205; A61B 5/00

(52) U.S. Cl. CPC G06F 19/3431; G06F 19/3481; A61B 5/486; A61B 5/4884; A61B 5/7225; A61B 5/7278; A61B 5/02055; A61B 5/6898; A61B 5/02405
USPC 702/19

(57) ABSTRACT

A health care system and method using human body indexes acquired from human body information are disclosed. The present health care method includes receiving human body information, calculating a human body index based on the received human body information, generating a health care program based on the calculated human body index and providing the generated health care program to a user. By this system and method, it becomes possible to measure and monitor stress/fatigue/health indexes of a user in living environments, thereby effectively supporting well-being life of the user.

### TECHNICAL FIELD

Methods and apparatuses consistent with the exemplary embodiments relate to a health care system and method, and more particularly, to a health care system which monitors and cares a physical condition of a user, and a method thereof.

### BACKGROUND ART

As people become more interested in health, various health care systems are emerging, but these health care systems are either terminals which merely measure the human body or apparatuses which simply measure and display users' health information.

Meanwhile, as people become more interested in stress which is regarded as the most threatening factor in health, many attempts are being made to measure stress, but mostly by medical institutions. However, these attempts disregard users' living environments where stress originates from, and thus cannot measure the exact stress.

Due to these problems, managing stress depends on subjective feelings of the users, where the users themselves check their physical conditions and search for solutions to resolve stress, and thus there is a limit in professionally managing stress.

### SUMMARY OF INVENTION

### 1. Solution to Problem

The purpose of the present disclosure is to solve the aforementioned problems, that is to provide a health care method and system which generates a health care program based on human body indexes calculated from human body information received real time, and to provide the generated health care program to a user.

### 2. Means for Solving Problems

An aspect of the exemplary embodiments relates to a health care method which may include receiving human body information; calculating a human body index, based on the human body information received at the step of receiving human body information; generating a health care program, based on the human body index calculated at the step of calculating a human body index; and providing the health care program generated at the step of generating a health care program, to a user.

In addition, the human body index may include at least one of a stress index, fatigue index, and health index.

In addition, the health index may be calculated based on the stress index and fatigue index.

In addition, it is desirable that the health care program includes at least one of exercise information and food information for health.

In addition, it is desirable that contents of the exercise information and food information for health differ according to the human body index.

In addition, it is desirable that the contents of the exercise information and food information for health differ discontinuously according to a section where the human body index belongs to.

In addition, the human body information may be HRV (Heart Rate Variation).

Meanwhile, according to an exemplary embodiment of the present disclosure, a health care system may include a measuring device which measures human body information; a communication module which receives human body information measured in the measuring device; and a user device which calculates a human body index based on the human body information received from the communication module, and generates a health care program based on the calculated human body index and provides the generated health care program to a user.

### 3. Effects of the Invention

As aforementioned, the present disclosure enables generating a health care program based on human body indexes calculated from human body information received real time, and providing the generated health care program to a user. Accordingly, it becomes possible to continuously measure and monitor a user's stress/fatigue/health indexes in living environments, thereby effectively supporting well-being life of the user.

In addition, it becomes possible to understand the degree of the user's stress/fatigue/health on a real time basis, and through a function of comparing them with the past, it becomes possible to provide feedback for the user's health care automatically. Furthermore, by comparing changed health conditions of the user and providing that information to the user, the present invention enables user-friendly and proactively health care, realizing active promotion of health.

### BRIEF DESCRIPTION OF THE DRAWINGS

The above and/or other aspects of the present disclosure will be more apparent by describing certain present disclosure with reference to the accompanying drawings, in which:

FIG. 1 is a block diagram of a health care system according to an exemplary embodiment of the present disclosure;

FIG. 2 is an exemplary table which lists stress indexes, fatigue indexes, and health indexes calculated by a smart phone for different health conditions and different age groups;

FIG. 3 is an exemplary table which lists a health care program generated/provided by a smart phone; and

FIG. 4 is a figure provided for explaining a health care method according to another exemplary embodiment of the present disclosure.

### DETAILED DESCRIPTION

Certain exemplary embodiments are described in higher detail below with reference to the accompanying drawings. In the following description, like drawing reference numerals are used for the like elements, even in different drawings. The matters defined in the description, such as detailed construction and elements, are provided to assist in a comprehensive understanding of exemplary embodiments. However, exemplary embodiments can be practiced without those specifically defined matters. Also, well-known functions or constructions are not described in detail since they would obscure the application with unnecessary detail.

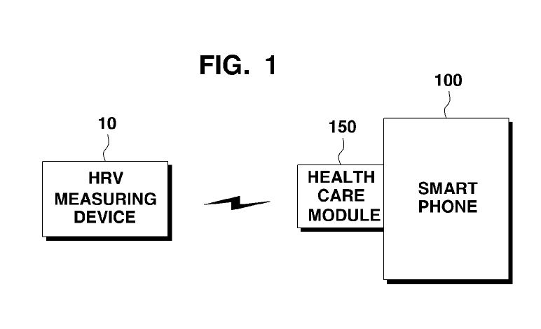

FIG. 1 is a block diagram of a health care system according to an exemplary embodiment of the present disclosure. The health care system according to an exemplary embodiment of the present disclosure measures HRV of a user, analyzes the measured HRV to calculate various body indexes, and generates a health care program based on the calculated body indexes to provide to the user.

As illustrated in FIG. 1, such a health care system which performs the aforementioned functions includes an HRV (Heart Rate Variation) measuring device 10, health care module 150, and smart phone 100, and may be embodied not only in a medical institution but also in a household. Not only that, analyzing the HRV, calculating body indexes, and generating and providing a health care program according to the health care system according to the exemplary embodiment may be made in real time.

The HRV measuring device 10 generates HRV data through measurement of heart beats of the user, and delivers the generated HRV data to the health care module 150. Delivering the HRV data from the HRV measuring device 10 to the health care module 150 may be performed through wireless communication in real time.

The health care module 150 is mounted on a smart phone 100, and is electrically connected to enable communication with the smart phone 100. Accordingly, the health care module 150 may deliver the HRV data to the smart phone 100. The delivery of the HRV data from the health care module 150 to the smart phone 100 is made in real time as well.

The smart phone 100 analyzes the HRV data received from the health care module 150, and calculates a stress index and fatigue index. In order to calculate the stress index and fatigue index, the smart phone 100 analyzes time domain characteristics and frequency domain characteristics regarding the HRV data.

In addition, based on the stress index and fatigue index, the smart phone 100 calculates a health index. More specifically, in calculating the health index, the smart phone 100 may give same/different weights to the stress index and fatigue index to calculate an average.

Meanwhile, based on the calculated stress index, the smart phone 100 may generate a health care program and provide it to the user by displaying it. The health care program provided to the user may include exercise information and food information for health.

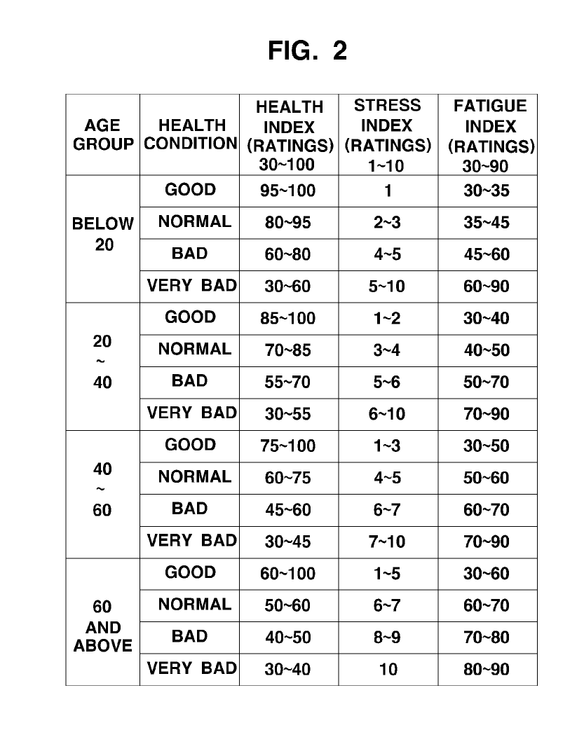

FIG. 2 is an exemplary table which lists stress indexes, fatigue indexes, and health indexes calculated by the smart phone 100 for different health conditions and different age groups. As illustrated in FIG. 2, the stress indexes, fatigue indexes, and health indexes are calculated in numerical values.

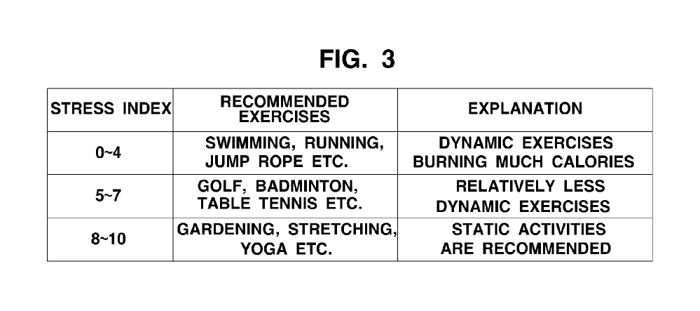

Meanwhile, FIG. 3 is an exemplary table which lists a health care program generated/provided by the smart phone 100. According to FIG. 3, different exercises are recommended for stress indexes, and more particularly, recommended exercises differ discontinuously according to the section where the stress index belongs to.

That is, it can be seen from FIG. 3, that different exercises are recommended according to whether the stress index belongs to “0˜4” section, “5˜7” section, or “8˜10” section.

In a case where the health care program is as illustrated in FIG. 3, and the user's stress index is calculated to be “6”, the smart phone 100 will recommend to the user “golf, badminton, table tennis etc.”, which are appropriate exercises for the stress index “6”.

Meanwhile, although not illustrated in FIG. 3, it is also possible to recommend appropriate food for different stress indexes, through the health care program.

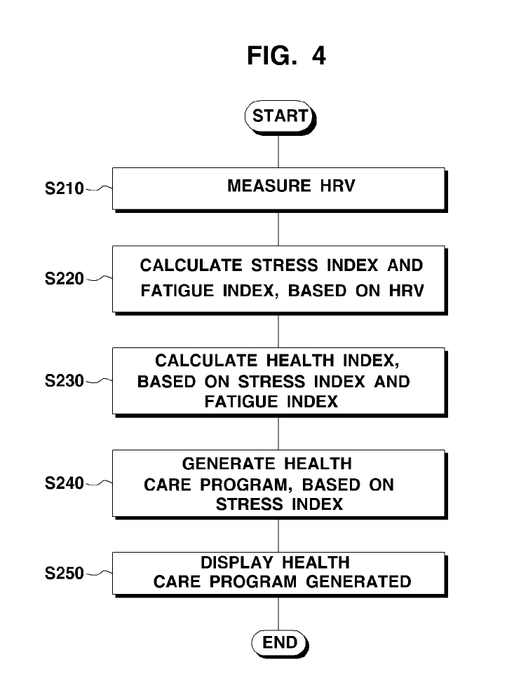

Hereinbelow is a more detailed explanation on a process of health care by the health care system illustrated in FIG. 1, with reference to FIG. 4. FIG. 4 is a figure provided to explain a health care method according to another exemplary embodiment of the present disclosure.

First of all, as illustrated in FIG. 4, the HRV measuring device 10 generates HRV data by measuring heart rates of the user (S210). The HRV data generated through S210 is delivered to the smart phone 100 through the health care module 150.

Then, the smart phone 100 analyzes the HRV data generated in S210, to calculate a stress index and fatigue index (S220).

Calculating the stress index in S220 is made through mathematical formula 1 as follows:

St-Lv=(A−B+C)*(LF_{—}nu/HF_{—}nu)/10 [Mathematical Formula 1]

A=Mo/SDNN

B=SDNN

C=(Mo/(HR_Max-HR_Min))/AMo

In the above mathematical formula 1, ‘St-Lv’ indicates the stress index which is calculated and shown as 1˜10. In addition, each variation is as follows:

LF_nu: LF(Low Frequency component) power normalization value

HF_nu: HF(High Frequency component) power normalization value

Mo: Mode value of heart rate

SDNN: Standard Deviation

HR_Max: Maximum heart rate value

HR_Min: Minimum heart rate value

Amo: (Mode value count/total data count)*100

Meanwhile, calculating fatigue index in S220 is made through mathematical formula 2 as follows:

Fa-Lv=274.652072−5.59950X+0.044447X²−0.000123X³ [Mathematical formula 2]

X=A+B

A=parasympathetic nerve index

B=HF_nu

In the above mathematical formula 2, ‘Fa=Lv’ is a fatigue index which is calculated and shown as 30˜90.

Next, the smart phone 100 uses the stress index and fatigue index calculated in S220 to calculate a health index (S230).

The calculation of a health index in S230 is made through mathematical formula 3 as follows:

Health-Lv=100−(((A*10+B)−40)/2) [Mathematical formula 3]

A=St-Lv(stress index)

B=Fa-Lv(fatigue index)

In the above mathematical formula 3, ‘Health-Lv’ is the health index which is calculated and shown as 30˜100.

In addition, the smart phone 100 generates a health care program based on the stress index calculated in S220 (S240). In addition, the smart phone 100 provides the user with the health care program generated in S240 by displaying it (S250).

It has been aforementioned that the health care program generated/provided in S250 may include exercise information and food information for health.

Meanwhile, in S250, it is possible to provide the stress index and fatigue index calculated in S220 and the health index calculated in S230 together with the health care program.

Aforementioned was the detailed explanation on a desirable exemplary embodiment of the health care system and method which measures the HRV of the user, calculates various human body indexes by analyzing the measured HRV, and generates a health care program based on the calculated human body indexes, and provides the generated health care program to the user.

In the above exemplary embodiment, the indexes are calculated through the HRV of the user, but this is merely an embodiment for convenience of explanation. Thus, it is possible to calculate the indexes through human body information other than HRV. Examples of other human body information include blood sugar, blood pressure, body temperature, and body weight etc.

In addition, the stress index, fatigue index, and health index mentioned in the above exemplary embodiment are also mere examples of human body indexes, and thus it is a matter of course that at least one of those indexes can be substituted for other human body indexes.

In addition, in the above exemplary embodiment, exercise information and food information are provided through the health care program, but it is also possible to embody the invention to provide other information for health as well.

In addition, in the above exemplary embodiment, the health care program is generated from the stress index, but the health care program may also be generated from other indexes as well.

Furthermore, it is also possible to embody the present invention to store the calculated human body indexes and the health care program in a database, and read and utilize the stored indexes and the health care program from the database.

Meanwhile, it is a matter of course that the present technological concept may be applied to a computer-readable record medium which includes computer programs which enable functions of the apparatus and method according to the exemplary embodiment of the present disclosure. In addition, the technological concept according to various exemplary embodiments of the present disclosure may be embodied into a computer-readable code format which is recorded in a computer-readable record medium. Any data storage device which may be read by a computer and may store data can be a computer-readable record medium. For example, a ROM, RAM, CD-ROM, magnetic tape, floppy disk, optical disk, hard disk drive etc. may be a computer-readable record medium. In addition, computer-readable codes or programs stored in a computer-readable record medium may be transmitted through an inter-computer network.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. A health care system comprising:
a measuring device configured to generate human body information of a user, wherein the human body information includes Heart Rate Variation (HRV), and wherein the HRV includes heart rate value and total count data of heart rates;
a user device configured to
receive the generated human body information from the measuring device through a health care module, wherein the health care module is mounted on the user device and wirelessly connected with the measuring device,
calculate a human body index, based on the received human body information,
generate a health care program which includes at least one of exercise information and food information for health, based on the calculated human body index, and
provide the generated health care program to the user,
wherein the human body index includes at least one of a stress index and fatigue index, and
wherein the stress index (St_{Lv}) is calculated by using the following equation:
St_{Lv}=(A<B+C)*(LFₙᵤ/HFₙᵤ)/10,
where
A=Mo/SDNN,
B=SDNN,
C=(Mo/(HR_{Max}−HR_{Min}))/AMo,
LFₙᵤ is a LF(Low Frequency component) power normalization value of the human body information,
HFₙᵤ is a HF(High Frequency component) power normalization value of the human body information,
Mo is a Mode value of heart rate value of the human body information,
SDNN is a Standard Deviation of the human body information,
HR_{Max} is a Maximum heart rate value of the human body information,
HR_{Min} is a Minimum heart rate value of the human body information, and
Amo=(Mo count of the human body information/total count data of the human body information)*100.

2. The health care system according to claim 1, wherein the human body index further includes health index which is calculated based on the stress index and the fatigue index.

3. The health care system according to claim 1, wherein contents of the exercise information and the food information for health differ according to the human body index.

4. The health care system according to claim 3, wherein the contents of the exercise information and the food information for health differ according to predetermined ranges of the human body index.

5. The health care system according to claim 1, wherein the human body information further includes at least one of blood sugar, blood pressure, body temperature, and body weight.

6. The health care system according to claim 1, wherein the fatigue index (Fa_{Lv}) is calculated by using the following equations:
Fa_{Lv}=274.652072−5.59950X+0.044447X²−0.000123X³, and
X=A+B,
where
A=parasympathetic nerve index of the human body information, and
B=HFₙᵤ.

7. The health care system according to claim 3, wherein the health index (Health_{Lv}) is calculated by using the following equation:
Health_{Lv}=100−(((A*10+B)−40)/2),
where
A=St_{Lv}, and
B=Fa_{Lv}.

8. The health care system according to claim 1, wherein the user device is a smart phone.

9. The health care system according to claim 1, wherein the health care program includes recommended exercises and explanations about the recommended exercises.
